(19) United States
(12) Patent Application Publication
(10) Pub. No.: US 2013/0143912 A1
(43) Pub. Date: Jun. 6, 2013

(54) SUBLINGUAL ZOLPIDEM FORMULATIONS

(71) Applicants: Dr. Reddy's Laboratories Limited, Hyderabad (IN); Dr. Reddy's Laboratories Inc., Bridgewater, NJ (US)

(72) Inventors: Snehalatha Movva, Hyderabad (IN); Natarajan Mathivanan, Coimbatore (IN); Priyanka Sriram Pathak, Jammu (IN); Ajay Kumar Narayan Sharma, Khargone (IN); Hemanth Prakash Joshi, Gadag (IN); Prasad Vure, Secunderabad (IN)

(73) Assignees: Dr. Reddy's Laboratories Inc., Bridgewater, NJ (US); Dr. Reddy's Laboratories Limited, Hyderabad (IN)

(21) Appl. No.: 13/705,683

(22) Filed: Dec. 5, 2012

(30) Foreign Application Priority Data
Dec. 6, 2011 (IN) IN 4240/CHE/2011

(51) Int. Cl. A61K 9/00

(52) U.S. Cl. CPC A61K 9/0002
USPC 514/300

(57) ABSTRACT

The present disclosure provides pharmaceutical compositions for the delivery of a hypnotic agent across the oral mucosa. In particular, the compositions devoid of buffer and in the presence of alkaline oxides capable of raising the pH of saliva to a pH greater than about 7.0 thereby facilitate the substantially complete conversion of the hypnotic agent from its ionized to its un-ionized form. As a result, the dose of hypnotic agent is rapidly and efficiently absorbed by the oral mucosa with surprisingly low inter-subject variability. Furthermore, delivery of the hypnotic agent across the oral mucosa advantageously bypasses hepatic first pass metabolism of the drug and avoids enzymatic degradation of the drug within the gastrointestinal tract. Methods for using the compositions of the present invention for treating sleep disorders such as insomnia are also provided.

The present application relates to rapidly acting pharmaceutical compositions for sublingual administration of a pharmaceutical agent and to methods for preparing such compositions.

The drug compound having the adopted name “zolpidem” has the chemical names: N,N-dimethyl-2-(6-methyl-2-p-tolylimidazo[1,2-a]pyridin-3-yl)acetamide; or N,N,6-trimethyl-2-p-tolylimidazo[1,2-a]pyridine-3-acetamide; and the structure below.

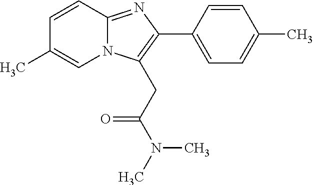

Zolpidem is a short-acting sedative that is used in the short-term management of insomnia. It has been demonstrated to reduce sleep latency, increase sleep duration, and reduce nighttime awakenings. In addition, zolpidem has been found to preserve stage III and stage IV sleep, and to result in less disruption of REM (rapid eye movement) sleep. The drug possesses a short half-life and produces no active metabolites. It appears to act by binding to the benzodiazepine receptor component of the GABA receptor complex and accordingly possesses similar properties to the benzodiazepines. However, zolpidem has the advantage of minimal anxiolytic, myorelaxant and convulsant properties.

Currently available zolpidem formulations contain doses of 5 and 10 mg of the drug in the form of its hemitartrate salt (see, for example, British National Formulary, Volume 48, pages 174 and 175). These compositions are administered orally, typically before retiring, and rapidly disintegrate in the gastrointestinal tract to provide systemic absorption of drug.

In the U.S. market, the AMBIEN CR® extended release tablets contain either 6.25 or 12.5 mg of zolpidem L-(+)-tartrate (2:1). The tablets have two layers, one releasing its drug content immediately and the other having a slower drug release.

A biphasic oral dosage form comprising zolpidem has been described in U.S. Pat. No. 6,514,531 B1. This dosage form has an initial immediate release phase to induce sleep as rapidly as possible, followed by a controlled-release phase with the objective of maintaining sleep following induction. Other biphasic tablets comprising zolpidem are disclosed in European Patent Application Publication No. 1 260 216 A1.

U.S. Pat. No. 6,638,535 B2 discloses sustained release pellets comprising short acting hypnotic agents, such as zolpidem, zopiclone, and zaleplon, which provide for an in vitro release of less than 60% of active ingredient within the first 5 minutes of the in vitro test.

International Patent Application Publication No. WO 00/16750 discloses a drug delivery system for the treatment of acute disorders by mucosal administration, in which the active ingredient is in micro-particulate form and is adhered to the surface of larger carrier particles in the presence of a bioadhesion and/or mucoadhesion promoting agent.

International Patent Application Publication No. WO 03/059349 discloses oral dosage forms comprising, inter alia, zolpidem, in addition to a solubility enhancer (e.g. a surfactant) and a spheronization agent (e.g. a distilled monoglyceride).

European Patent No. 0 324 725 describes technology for formulating rapidly dissolving ordered-mixture compositions in which the drug in a finely dispersed state covers the surface of substantially larger carrier particles. Such compositions disintegrate rapidly in water, thereby dispersing their contents of microscopic drug particles.

Oral administration, however, has several disadvantages, such as drug losses during hepatic first pass metabolism, during enzymatic degradation within the gastrointestinal tract, and during absorption. This leads to greater variability in response, and necessitates larger initial doses. For example, in view of the first-pass and/or pre-systemic metabolism that is typically connected with oral administration, the use of currently-marketed zolpidem formulations is characterised by considerable inter- and intra-individual variability in terms of both onset of action and residual effects, See, for example, Holm et al, Drugs (2000) 59, 865; Darcourt et al, J. Pharmacol., (1999) 13, 81; Terzano et al, Drug Safety (2003) 26, 261; Salva and Costa, Clin. Pharmacokinet. (1995) 29, 142; Drover et al, Clin. Ther. (2000) 22, 1443; and “Guidance for Industry; Labeling Guidance for Zolpidem Tablets”, US Department of Health and Human Services (1997).

In addition, because the drug has to pass through the gastrointestinal system in order to enter the blood stream, the time to achieve a therapeutic effect may be quite long, typically around 45 minutes or longer. In view of this, onset of action can be delayed in many patients, leading to a frustrating lack of “on demand” sleep, and possibly to undesirable residual effects the following day. Accordingly, other routes of drug administration have been explored, including those involving transport across the mucous membranes. Of the various mucous membranes (e.g., oral, rectal, vaginal, ocular, nasal, etc.), drug delivery via the mucous membranes in the oral cavity is usually preferred. In addition to avoiding the problems with traditional oral ingestion, drug delivery via the mucous membranes of the oral cavity has other advantages as the mucous membranes of the oral cavity are highly vascularized and well supplied with lymphatic drainage sites.

It might be expected that transmucosal administration of an active ingredient would result in an enhanced rate of absorption as compared to an oral ingestion, and thereby result in a vastly increased bioavailability in a short period of time. In the treatment of insomnia with a short-acting hypnotic agent such as zolpidem, such an enhanced rate of absorption might be expected to cause potential safety problems in patients sensitive to the drug, giving rise to undesirable pharmacological effects, such as a more rapid onset of sleep than is convenient (e.g. when preparing for sleep; see, for example column 2, lines 9 to 18 of U.S. Pat. No. 6,638,535 B2). Moreover, such a rapid absorption may be expected to compromise the duration of action of the drug, and thereby affecting the ability to maintain sleep during the night, especially since short-acting compounds are known to be rapidly eliminated from plasma (see, for example, column 2, lines 19 to 31 of U.S. Pat. No. 6,638,535 B2).

In general, the mucous membranes of the oral cavity can be divided into five main regions: sublingual (floor of the mouth), buccal (cheeks), gingival (gums), palatal (roof of the mouth), and the lining of the lips. These regions differ from each other with respect to their anatomy, drug permeability, and physiological response to drugs. For example, sublingual is more permeable than buccal, which is more permeable than palatal. Permeability depends on the relative thickness and degree of keratinization of the membranes, with the sublingual mucosa being relatively thin and non-keratinized, the buccal mucosa being thicker and non-keratinized, and the palatal mucosa being intermediate in thickness, but keratinized.

In addition to the differences in permeability of the various mucous membranes, the extent of drug delivery is also affected by the properties of the drug to be delivered. The ability of a molecule to pass through any mucous membrane is dependent upon its size, its lipid solubility, and the extent to which it is ionized, among other factors. The extent to which a drug is ionized is dependent on the dissociation constant (pKₐ), and the pH of the molecule's surrounding environment. In its non-ionized form, a drug is sufficiently lipophilic to traverse a membrane via passive diffusion. In fact, according to the pH partition hypothesis, only non-ionized, non-polar drugs will penetrate a lipid membrane. Maximum absorption across the membrane is thought to occur when a drug is 100% in its non-ionized form. Similarly, absorption across the membrane decreases as the extent of ionization increases. Therefore, one may influence the extent of drug absorption across the mucous membranes of the oral cavity by altering the salivary pH.

There is a need for improved pharmaceutical formulations, capable of delivering hypnotic agents across the oral mucosa with a rapid onset of action and acceptable residual effects.

### SUMMARY

Aspects of the present application are directed to sublingual formulations comprising zolpidem wherein a dosage unit of the pharmaceutical composition is placed under tongue, and the active component is absorbed through the surrounding mucous membranes.

In aspects, the application relates to single-dose pharmaceutical formulations for sublingual administration, comprising a pharmacologically effective amount of at least one pharmaceutically active agent.

In aspects, the application relates to pharmaceutical formulations for sublingual administration that can be used for the treatment of acute disorders.

In aspects, the application provides methods for making such formulations.

In accordance with aspects of the application, pharmaceutical formulations comprise pharmaceutically effective amounts of zolpidem or any of its salts, together with a buffer or combination of buffers.

In another aspect of the application, pharmaceutical formulations comprise pharmaceutically effective amounts of zolpidem or any of its salts, devoid of a buffer or combination of buffers.

In another aspect of the application, pharmaceutical formulations comprise pharmaceutically effective amounts of zolpidem or any of its salts, together with a pH inducing agent or alkaline oxides.

In aspects, the formulations of the present application are in the form of sublingual tablets comprising zolpidem, a buffer, and any other excipients.

In aspects, the formulations of the present application are in the form of sublingual tablets comprising zolpidem, a buffer, and a carrier, providing complete buccal or sublingual disintegration in about 5 minutes or less following placement in a person's mouth.

In aspects, formulations of the present application undergo dissolution in the oral cavity in at least about 5 minutes or more.

In aspects, the formulations of the application can be used in the treatment of acute disorders such as insomnia.

### DETAILED DESCRIPTION

In aspects, the present application is directed to sublingual formulations of zolpidem that can be used for the treatment of acute disorders such as insomnia.

In embodiments, formulations comprise pharmaceutically effective amounts of zolpidem or any of its salts, together with a buffer or combination of buffers.

In embodiments, a buffer is a salt of carbonic acid, a bicarbonate salt, a basic amino acid, or an amino sugar such as meglumine.

In embodiments, a buffer is a combination of two or more buffering agents that act to raise the pH of the saliva higher than 7.

In embodiments, formulations of the present application are in the form of sublingual tablets comprising zolpidem, a buffer, and a carrier, providing substantially complete buccal or sublingual disintegration in about 5 minutes or less following administration to the mouth.

In embodiments, a carrier comprises at least one binder and at least one disintegrating agent in suitable proportions to provide a sublingual disintegration time of about 5 minutes or less, or about 2 minutes or less, following administration to the mouth.

In embodiments, formulations of the present application are dosage forms such as lozenges, chewing gums, chewable tablets, and dissolving tablets such as slow-dissolving tablets or quick-dissolving tablets.

The term “zolpidem” as used herein refers to any form of zolpidem such as a salt form, a free base form, a polymorphic form, or any mixtures thereof. For example, pharmaceutically acceptable salts of zolpidem include, without limitation, tartrate, hemitartrate, succinate, dihydrochloride, salicylate, hemisuccinate, citrate, maleate, hydrochloride, carbamate, sulfate, nitrate, and benzoate salt forms, as well as combinations thereof. In some embodiments, the zolpidem is a hemitartrate.

The pharmaceutical formulations of the present application may, in addition to zolpidem, also contain one or more additional formulation ingredients chosen from a wide variety of excipients known in the art. According to the desired properties of the formulation, any number of ingredients may be used, alone or in combination, based upon their functions. Such ingredients include, but are not limited to, buffers, diluents, binders, disintegrants, compression aids, lubricants, flavors, sweeteners, colorants, and preservatives.

In embodiments, the present application is directed to sublingual pharmaceutical formulations comprising zolpidem, a buffer, a binder, a disintegrant, and a lubricant, and optionally one or more additional agents such as flavoring agents, colorants, and sweeteners.

In embodiments, the present application is directed to disintegrating formulations comprising zolpidem as an active ingredient, a superdisintegrant such as croscarmellose, crospovidone, or sodium starch glycolate, a buffer such as sodium carbonate or meglumine, and other desired excipients.

In embodiments, pharmaceutical formulations of the present application have a sublingual disintegration time of about 5 minutes or less, or about 2 minutes or less, following administration to the mouth.

In embodiments, formulations of the present application undergo disintegration within about 2 -10 minutes, or about 5 minutes, or about 7 minutes, following administration to the mouth.

Typically, the formulations of the present application comprise less than about 5 mg (e.g., from about 0.5 mg to about 4.75 mg, from about 1.5 mg to about 2.5 mg, from about 3.0 mg to about 3.75 mg, etc.) of zolpidem or a salt thereof. One skilled in the art understands that the amounts will vary depending upon the particular source of zolpidem utilized, the amount of zolpidem desired in the final formulation, and the particular release rate of zolpidem desired. In certain embodiments, the use of buffer in the compositions provides a final salivary pH in excess of at least about 7, or at least about 7.8, or at least about 8.5, or at least about 9 (e.g., about 9-11). In embodiments, the buffer or combination of buffers raises the pH of saliva to values greater than about 7.2, 7.6, 7.8, 8, 8.3, 8.5, or 8.8. In embodiments, the buffer or combination of buffers raises the pH of saliva to values greater than about 9, 9.4, 9.5, 9.6, 9.7, or 9.8 (e.g., about 9-11).

As used herein, the pH inducing agent or alkaline oxide refers to a suitable metal oxides include potassium oxide, sodium oxide, barium oxide, magnesium oxide and aluminum oxide.

Suitable citrate, phosphate, and borate salts include, without limitation, any salt of citric acid, phosphoric acid, or boric acid known in the art such as those described above. In certain instances, the binary buffer system comprises a metal oxide and a citrate salt. In certain other instances, the binary buffer system comprises a metal oxide and a phosphate salt. In further instances, the binary buffer system comprises a metal oxide and a borate salt.

As used herein, a buffer or buffering agent is a weak base, used to maintain the pH of a solution at a desired value. Useful buffers of the present application include, but are not limited to, carbonate salts, bicarbonate salts, basic amino acids, amino sugars, and any combinations of two or more thereof. For example, a carbonate salt can be sodium carbonate, potassium carbonate, calcium carbonate, ammonium carbonate, or magnesium carbonate. A bicarbonate salt can be sodium bicarbonate, potassium bicarbonate, calcium bicarbonate, ammonium bicarbonate, or magnesium bicarbonate. A basic amino acid can be arginine, histidine, lysine, glycine, etc. An amino sugar may be N-acetylglucosamine, galactosamine, glucosamine, sialic acid, or meglumine. In embodiments, the buffer comprises a combination of meglumine and glycine.

Formulations of the present application can be prepared using conventional methods that are well known in the art. Granules can be formed by any processes, using operations such as one or more of dry granulation, wet granulation, extrusion-spheronization, and the like. In embodiments, a granulating fluid that is used is non-aqueous.

In embodiments, granulation of the active ingredient, optionally together with one or more pharmaceutically acceptable excipients such as diluents or fillers, may be carried out in equipment such as planetary mixers, double cone blenders, rapid mixer granulators (RMG), fluid bed processors, and the like. Alternatively, powder blends may be compacted using a roller compactor and then milled to produce granules that are suitable for compression. The granules obtained may further be compressed into tablets or filled into capsules, using techniques known in the art.

Alternatively, tablets may be prepared by a direct compression technique, using powder blends. Alternatively, other technologies known in the art, such as freeze-drying, spray drying, mass extrusion and molding, may also be used to prepare formulations in accordance with the present application.

In the context of the present application, during the processing of pharmaceutical compositions into finished dosage forms, one or more pharmaceutically acceptable excipients may optionally be included, such as but not limited to any one or more of diluents, binders, disintegrants, lubricants, glidants, coloring agents, wetting agents, film-forming agents, emulsifying agents; solubilizing agents; suspending agents, and preserving agents such as methyl-, ethyl-, and propyl-hydroxybenzoates, butylated hydroxytoluene, and butylated hydroxyanisole and others.

Various useful fillers or diluents include, but are not limited to, starches, lactose, cellulose derivatives, confectioner's sugar, and the like. Different grades of lactose include, but are not limited to, lactose monohydrate, lactose DT (direct tableting), lactose anhydrous, Flowlac™ (available from Meggle Products), Pharmatose™ (available from DMV), and others. Different starches include, but are not limited to, maize starch, potato starch, rice starch, wheat starch, pregelatinized starch (commercially available as PCS PC10 from Signet Chemical Corporation) and starch 1500, starch 1500 LM grade (low moisture content grade) from Colorcon, fully pregelatinized starch (commercially available as National 78-1551 from Essex Grain Products), and others. Different cellulose compounds that can be used include crystalline cellulose and powdered cellulose. Examples of crystalline cellulose products include, but are not limited to, Ceolus™ KG801, Avicel™ PH101, PH102, PH301, PH302 and PH-F20, PH-112 microcrystalline cellulose 114, microcrystalline cellulose 112, and silicified microcrystalline cellulose (e.g., Prosolv198 supplied by JRS Pharma). Other useful diluents include, but are not limited to, carmellose, sugar alcohols such as mannitol (e.g., Pearlitol™ SD200), sorbitol, and xylitol, calcium carbonate, magnesium carbonate, dibasic calcium phosphate, and tribasic calcium phosphate.

Suitable carriers for use in the formulations of the present application include, without limitation, binders, gum bases, and combinations thereof. Non-limiting examples of binders include mannitol, sorbitol, xylitol, maltodextrin, lactose, dextrose, sucrose, glucose, inositol, powdered sugar, molasses, starch, cellulose, microcrystalline cellulose, polyvinylpyrrolidones, acacia gum, guar gum, tragacanth gum, alginate, extract of Irish moss, panwar gum, ghatti gum, mucilage of isapol husks, Veegum™, larch arabogalactan, gelatin, methylcelluloses, ethylcelluloses, carboxymethylcelluloses, hydroxypropyl methylcelluloses, polyoxyethylene polymers, polyacrylic acids (e.g., Carbopol® products), calcium silicate, calcium phosphate, dicalcium phosphate, calcium sulfate, kaolin, sodium chloride, polyethylene glycols, propylene glycols, and any combinations thereof. These binders can be pre-processed to improve their flowability and taste by methods known in the art such as freeze drying (see, e.g., Fundamentals of Freeze-Drying, Pharm. Biotechnol., 14:281-360 (2002); Lyophililization of Unit Dose Pharmaceutical Dosage Forms, Drug. Dev. Ind. Pharm., 29:595-602 (2003)); solid-solution preparation (see, e.g., U.S. Pat. No. 6,264,987); and lubricant dusting and wet-granulation preparation with a suitable lubricating agent (see, e.g., Remington: The Science and Practice of Pharmacy). For example, Mannogem™ and Sorbogem™, sold by SPI Pharma Group (New Castle, Del.), are freeze-dried processed forms of mannitol and sorbitol, respectively. Typically, the compositions of the present application comprise from about 25% to about 90% by weight of the binder, or from about 50% to about 80%.

Various useful disintegrants include, but are not limited to, carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™ from FMC-Asahi Chemical Industry Co., Ltd.), crospovidones, examples of commercially available crospovidone products including but not limited to crosslinked povidone, Kollidon CL from BASF (Germany), PolyplasdoneTM XL, XI-10, and INF-10 from

ISP Inc. (USA), and low-substituted hydroxypropylcelluloses. Examples of low-substituted hydroxypropylcelluloses include, but are not limited to, low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

In certain embodiments, superdisintegrants may be used to provide rapid disintegration of the formulation on contact with aqueous fluids. Superdisintegrants provide quick disintegration due to combined effect of swelling and water absorption by the formulation. Non-limiting examples of superdisintegrants are croscarmellose (e.g., Ac-Di-Sol™ from FMC BioPolymer), crospovidones (e.g., Kollidon® CL, Kollidon CL-F, Kollidon CL-SF, and Kollidon CL-M from BASF), and sodium starch glycolate.

An effective amount of any pharmaceutically acceptable tableting lubricant can be added to assist with compressing tablets and improving particle flow characteristics. Useful tablet lubricants include magnesium stearate, glyceryl monostearate, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and any combinations thereof.

One or more glidant materials, which improve the flow of powder blends and minimize dosage form weight variation, can be used. Useful glidants include, but are not limited to, silicon dioxide, talc, and combinations thereof.

The compositions of the present application may optionally contain a coloring agent. Suitable coloring agents include, without limitation, natural and/or artificial materials such as FD&C coloring agents, natural juice concentrates, pigments such as titanium oxide, iron oxides, silicon dioxide, and zinc oxide, combinations thereof, and the like.

Equipment suitable for processing pharmaceutical compositions of the present application include rapid mixer granulators, planetary mixers, double cone blenders, mass mixers, ribbon mixers, fluid bed processors, mechanical sifters, homogenizers, blenders, roller compacters, extrusion-spheronizers, compression machines, capsule filling machines, rotating bowls or coating pans, tray dryers, fluid bed dryers, rotary cone vacuum dryers, and the like, multi-mills, fluid energy mills, ball mills, colloid mills, roller mills, hammer mills, and the like, equipped with a suitable screen.

The pharmaceutical dosage forms of the present application are intended for sublingual administration to a patient in need thereof.

The pharmaceutical formulations of the present application are useful in the therapeutic or prophylactic treatment of acute disorders, in patients suffering from sleep disorders such as insomnia. The compositions of the present application can be used to treat insomnia prophylactically.

The following examples are presented to further illustrate various specific aspects and embodiments of the present application, but are not intended to limit the scope of the application in any respect.

### Defintions

As used herein, the following terms have the meanings ascribed to them unless specified otherwise.

As used herein, the term “insomnia” refers to a sleep disorder characterized by symptoms including, without limitation, difficulty in falling asleep, difficulty in staying asleep, intermittent wakefulness, and/or waking up too early. The term also encompasses daytime symptoms such as sleepiness, anxiety, impaired concentration, impaired memory, and irritability. Types of insomnia suitable for treatment with the compositions of the present application include, without limitation, transient, short-term, and chronic insomnia. The term “transient insomnia” refers to insomnia lasting for a few nights. The term “short-term insomnia” refers to insomnia lasting for about two to about four weeks. The term “chronic insomnia” refers to insomnia lasting for at least one month.

As used herein, the term “therapeutically effective amount” or “effective amount” refers to the amount of zolpidem that is capable of achieving a therapeutic effect in a subject in need thereof. For example, an effective amount of zolpidem can be the amount that is capable of preventing or relieving one or more symptoms associated with insomnia.

As used herein, the term “disintegrates” or “disintegration” refers to the breakdown of, for example, a tablet or lozenge, into small particles accompanied by dissolution of a substantial portion of the active pharmaceutical ingredient. More particularly, disintegration of a pharmaceutical composition refers to less than about 25% by weight of the pharmaceutical composition remaining in the mouth following an appropriate time period, e.g., about 5 minutes after administration. Suitable methods known in the art for determining the disintegration profile of a pharmaceutical composition include, e.g., test 701 “Disintegration” from United States Pharmacopeia 29, United States Pharmacopeial Convention, Inc., Rockville, Md., 2005.

As used herein, the term “treating” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom or condition. The treatment or amelioration of symptoms can be based on any objective or subjective parameter; including, e.g., the result of a physical examination.

### EXAMPLES

### Example 1

### Zoplidem Sublingual Ttablets

| Ingredient | mg/Tablet |
|---|---|
| Zolpidem tartrate | 3.5 |
| Mannitol | 99.6 |
| Sorbitol | 6.3 |
| Crospovidone | 27.6 |
| Colloidal silicon dioxide | 1 |
| Meglumine | 24 |
| Glycine | 33 |
| Flavor | 6.5 |
| Sucralose | 1.5 |
| Color | 1 |
| Magnesium stearate | 6 |

Manufacturing procedure:

1. Zolpidem tartrate and colloidal silicon dioxide are co-sifted through a #40 mesh sieve.

2. Mannitol, sorbitol, crospovidone, meglumine, glycine, flavor, and sucralose are sifted through a #40 mesh sieve.

3. Ingredients of 1 and 2 are blended.

4. Color and magnesium stearate are co-sifted through a #60 mesh sieve, and blended with the mixture of 3.

5. The blend of 4 is compressed into tablets.

### Example 2

### Zolpidem Sublingual Tablets

| Ingredient | mg/Tablet |
|---|---|
| Zolpidem tartrate | 3.5 |
| Mannitol | 132.3 |
| Sorbitol | 7.9 |
| Crospovidone | 26.3 |
| Colloidal silicon dioxide | 1 |
| Meglumine | 24 |
| Flavor | 6.5 |
| Sucralose | 1.5 |
| Color | 1 |
| Magnesium stearate | 6 |

Manufacturing procedure:

1. Zolpidem tartrate and colloidal silicon dioxide are co-sifted through a #40 mesh sieve.

2. Mannitol, sorbitol, a portion of the crospovidone, meglumine, glycine, flavor, and sucralose are sifted through a #40 mesh sieve.

3. Ingredients of 1 and 2 are blended.

4. Color and a portion of the magnesium stearate are co-sifted through a #60 mesh sieve and blended with the mixture of 3.

5. The blend of 4 is compressed into tablets having hardness of 2-3 kiloponds (kp).

6. Tablets of 5 are passed through a Quadro® Comil® conical mill to form granules.

7. The remaining portions of crospovidone and magnesium stearate are sifted through a #40 mesh sieve and blended with the granules of 6.

8. The blend of 7 is compressed into tablets.

### Example 3

### Zolpidem Sublingual Tablets

| Ingredient | mg/Tablet |
|---|---|
| Zolpidem tartrate | 1.75 |
| Mannitol | 129.05 |
| Sorbitol | 7.6 |
| Crospovidone | 25.6 |
| Colloidal silicon dioxide | 1 |
| Magnesium oxide light | 30 |
| Flavor | 6.5 |
| Sucralose | 1.5 |
| Color | 1 |
| Magnesium stearate | 6 |

Manufacturing procedure:

1. Zolpidem tartrate and colloidal silicon dioxide are co-sifted through a #40 mesh sieve.

2. Mannitol, sorbitol, a portion of the crospovidone, magnesium oxide, flavor, and sucralose are sifted through a #40 mesh sieve.

3. Ingredients of 1 and 2 are blended together.

4. Color and a portion of the magnesium stearate are co-sifted through a #60 mesh sieve, added to the mixture of 3, and blended.

5. The blend of 4 is compressed into tablets having hardness of 2-3 kp.

6. The tablets are passed through a Quadro Comil to form granules.

7. Remaining portions of the crospovidone and magnesium stearate are sifted through a #40 mesh sieve and blended with the granules of 6.

8. The blend of 7 is compressed into tablets.

### Examples 4-8

### Zolpidem Sublingual Tablets

The compositions of the tables below are processed using procedures similar to those of any of the preceding examples.

| | mg/Tablet | | | | | |
|---|---|---|---|---|---|---|
| Ingredient | 4A | 4B | 5A | 5B | 6A | 6B |
| Zolpidem tartrate | 3.5 | 1.75 | 3.5 | 1.75 | 3.5 | 1.75 |
| Sodium bicarbonate | 40 | 40 | — | — | | — |
| Calcium bicarbonate | — | — | 40 | 40 | — | — |
| Arginine, lysine, or histidine | — | — | — | — | 40 | 40 |
| Pharmaburst B2* | 139.5 | 141.25 | — | — | 139.5 | 141.25 |
| Ludiflash** | — | — | 139.5 | 141.25 | — | — |
| Croscarmellose sodium or | 10 | 10 | 10 | 10 | 10 | 10 |
| crospovidone | | | | | | |
| Natural & artificial spearmint flavor | 6.5 | 6.5 | 6.5 | 6.5 | 6.5 | 6.5 |
| Sucralose | 1.5 | 1.5 | 1.5 | 1.5 | 1.5 | 1.5 |
| Silicon dioxide | 5.5 | 5.5 | 5.5 | 5.5 | 5.5 | 5.5 |
| Color | q.s. | q.s. | q.s. | q.s. | q.s. | q.s. |
| Flavor | q.s. | q.s. | q.s. | q.s. | q.s. | q.s. |
| Magnesium stearate | 3.5 | 3.5 | 3.5 | 3.5 | 3.5 | 3.5 |

*PHARMABURST B2 is 85 percent mannitol, 10 percent polyplasdone, 5 percent sorbitol, and less than one percent hydrated colloidal silica, sold by SPI Pharma.
**LUDIFLASH is a co-processed blend of 90% mannitol, 5% crospovidone, and 5% polyvinyl acetate dispersion, stabilized with povidone, sold by BASF.

| | mg/Tablet | | | |
|---|---|---|---|---|
| Ingredient | 7A | 7B | 8A | 8B |
| Zolpidem tartrate | 3.5 | 1.75 | 3.5 | 1.75 |
| Sodium carbonate | — | — | 30 | 30 |
| Sodium bicarbonate | — | — | 15 | 15 |
| Sodium phosphate dibasic | 30 | 30 | 36 | 36 |
| Sodium phosphate tribasic | 15 | 15 | — | — |
| Pharmaburst B2 | 134.5 | 136.25 | 98.5 | 100.25 |
| Croscarmellose sodium or | 10 | 10 | 10 | 10 |
| crospovidone | | | | |
| Natural & artificial spearmint flavor | 6.5 | 6.5 | 6.5 | 6.5 |
| Sucralose | 1.5 | 1.5 | 1.5 | 1.5 |
| Silicon dioxide | 5.5 | 5.5 | 5.5 | 5.5 |
| Color | q.s. | q.s. | q.s. | q.s. |
| Flavor | q.s. | q.s. | q.s. | q.s. |
| Magnesium stearate | 3.5 | 3.5 | 3.5 | 3.5 |

### Example 9

### Zolpidem Sublingual Tablets

| | | 1.75 mg | 3.5 mg |
|---|---|---|---|
| S. | | Quantity | Quantity |
| No. | Ingredients | (mg/tab) | (mg/tab) |
| 1 | Zolpidem Tartrate, USP | 1.75 | 3.50 |
| 2 | Mannitol, USP (Pearlitol 200 SD) (Part 1) | 75.00 | 75.00 |
| 3 | Mannitol, USP (Pearlitol 200 SD) (Part 2) | 76.45 | 74.70 |
| 4 | Magnesium Oxide Light, USP | 15.00 | 15.00 |
| 5 | Sorbitol, NF (Neosorb P 100T) | 6.30 | 6.30 |
| 6 | Croscarmellose Sodium, NF (Ac-Di- Sol) | 10.00 | 10.00 |
| 7 | Ferric oxide, NF (Iron oxide yellow) | 1.00 | — |
| 8 | Pigment blend (PB-1346 Yellow) | — | 1.00 |
| 9 | Spearmint Flavor(N-C 913.004) | 3.00 | 3.00 |
| 10 | Sucralose, NF | 1.50 | 1.50 |
| 11 | Colloidal Silicon Dioxide, NF(Aerosil | 2.00 | 2.00 |
| | 200) | | |
| 12 | Silicon Dioxide(Syloid 244 FP) | 8.00 | 8.00 |
| 13 | Sodium Stearyl fumarate, NF(Pruv) | 10.00 | 10.00 |

| | | |
|---|---|---|
| Total Weight | 210.00 | 210.00 |

Manufacturing Process

Sifting:

Sift the materials, Ferric oxide Yellow, NF /Pigment Blend (PB-1346 Yellow), IH and Spearmint Flavor (N-C 913.004),IH together using #80 mesh and collect the materials in suitable labeled HDPE drum lined with double polyethylene bag.

Pre-Mix:

Load the Mannitol part 1, USP (Part 1) into the Double Cone Blender. Blend it for 2 minutes at blender rpm: 23 rpm (22 rpm-24 rpm). Load the sifted material from Step #1.1, into the Double Cone Blender, followed by Zolpidem Tartrate, USP, Sorbitol, NF (rinsed with API bag), Croscarmellose Sodium, NF, Sucralose, NF, Colloidal Silicon Dioxide, NF, Silicon Dioxide, NF, Magnesium Oxide Light, USP, and Finally add Mannitol, USP (Part 2) and blend for 15 minutes at blender rpm: 23 rpm (22 rpm-24 rpm). Unload the pre-mix blend from Step #1.2 into HDPE containers lined with double polyethylene bags.

Sifting:

Sift the materials, from Step #1.3 using #40 meshes and collect the materials in suitable labeled HDPE drum lined with double polyethylene bag.

Blending:

Reload the above sifted blend in the Double Cone Blender, blend for 15 minutes at blender rpm 23 rpm (22rpm-24rpm).

Lubrication/Final Mix:

Sodium Stearyl Fumarate, NF sifted through #60 mesh and load to the blender and blend for 15 minutes at 23 rpm (22 rpm-24 rpm)

Compression:

Compress the lubricated blend by using 9.5 mm shallow concave punches embossed with T2 for 1.75mg tablet and T3 for 3.5 mg tablet.

Packaging:

Compressed tablets packed in bulk packs (Triple laminated polybags—1 kg Tablets) and sent to Reed lane. Blister 10's packaging at Reedlane.10′s blister pack cut in 1's blister at Reedlane.1's blister filled in foil pouch and kept in cartons 50's pack.

Throughout this application, various publications are referenced. The disclosures of these publications in their entireties are hereby incorporated by reference into this application in order to more fully describe the state of the art as known to those skilled therein as of the date of the application described.

While particular embodiments of the present specification have been illustrated and described, it would be apparent to those skilled in the art that various other changes and modifications can be made without departing from the spirit and scope of the application. It is therefore intended to cover such changes and modifications that are within the scope of this application.

We claim:

1. A pharmaceutical composition comprising zolpidem in an amount from about 1 mg to about 20 mg, and at least one pH inducing agent and free of buffer, wherein zolpidem is absorbed across the subject's oral mucosa, wherein pH inducing agent is capable of raising the pH of saliva to a pH of about 7.0 or greater, and wherein at least 75% of the solid pharmaceutical composition dissolves within about 10 minutes or less within an oral cavity following administration.

2. A pharmaceutical composition comprising zolpidem in an amount from about 1 mg to about 20 mg, and at least one alkaline oxides and free of buffer, wherein zolpidem is absorbed across the subject's oral mucosa, wherein alkaline oxide is capable of raising the pH of saliva to a pH of about 7.5 or greater, and wherein at least 75% of the solid pharmaceutical composition dissolves within about 10 minutes or less within an oral cavity following administration.

3. The pharmaceutical composition of claim 2, wherein the solid pharmaceutical composition further comprises a binder and a disintegrating agent.

4. The pharmaceutical composition of claim 2, wherein the oral mucosa is selected from the group consisting of sublingual mucosa, buccal mucosa, gingival mucosa, palatal mucosa, and lining of the lips.

5. The pharmaceutical composition of claim 2, wherein a mean peak plasma concentration of zolpidem between about 20 to about 100 ng/mL is produced within about 30 minutes.

6. The pharmaceutical composition of claim 2, wherein a therapeutically effective amount of zolpidem enters the bloodstream within about 30 minutes.

7. The pharmaceutical composition of claim 2, wherein the solid pharmaceutical composition is a lozenge or tablet.

8. The pharmaceutical composition of claim 2, wherein the solid pharmaceutical composition is a lozenge or tablet.

9. The pharmaceutical composition of claim 2, wherein the solid pharmaceutical composition contains at least one pH inducing agent selected from magnesium oxide, potassium oxide, calcium oxide, aluminium oxide or combinations thereof.

10. The pharmaceutical composition of claim 2, for oral administration, wherein the solid pharmaceutical composition contains atleast one pH inducing agent, mannitol, disintegrant, and sorbitol or combinations thereof.
